(19) United States
(12) Patent Application Publication
(10) Pub. No.: US 2025/0101415 A1
(43) Pub. Date: Mar. 27, 2025

(54) DNA ORIGAMI STRUCTURE AND PROTEIN NANOPORE CONSTRUCT

(71) Applicant: Illumina, Inc., San Diego, CA (US)

(72) Inventors: Miguel Angel Aleman Garcia, Cambridge (GB); Alexandre Richez, Cambridge (GB)

(21) Appl. No.: 18/575,265

(22) PCT Filed: Apr. 24, 2023
(86) PCT No.: PCT/US2023/019596

§ 371 Date: Dec. 28, 2023

Related U.S. Application Data
(60) Provisional application No. 63/363,634, filed on Apr. 26, 2022.

(51) Int. Cl. C12N 15/11; B82Y 5/00; C12Q 1/6869

(52) U.S. Cl. CPC C12N 15/11; C12Q 1/6869; B82Y 5/00; C12N 2310/3515; C12N 2310/50

(57) ABSTRACT

The present application discloses a novel DNA origami structure and a nanopore construct associated with the DNA origami structure. The DNA origami structure includes a first hydrophilic section at a first end of the DNA origami structure, a stopper section adjacent the first hydrophilic section, a second hydrophilic section at a second end of the DNA origami structure, a hydrophobic section between the stopper section and the second hydrophilic section, and an open cavity running through the DNA origami structure from the first end to the second end. The stopper section is configured to lay against the membrane when the DNA origami structure is inserted through the membrane.

### INCORPORATION BY REFERENCE TO ANY PRIORITY APPLICATIONS

Any and all applications for which a foreign or domestic priority claim is identified in the PCT Application as filed with the present application are hereby incorporated by reference.

### FIELD OF THE INVENTION

Some embodiments provided herein include methods and compositions for sequencing single stranded DNA. Some embodiments include a novel DNA origami structure. In some embodiments, the DNA origami structure is incorporated into a membrane.

### BACKGROUND OF THE INVENTION

DNA sequencing is the identification of the order of nucleotides within a strand of DNA. Our progressive understanding of DNA has been indispensable towards a wide range of new therapeutic avenues, including cancer therapeutics, personalized medicine, and treatment of genetic disorders.

DNA sequencing on patterned flowcells is becoming the standard for many DNA sequencers at Illumina. However, despite delivering very high-quality data, those sequencers only support relatively short read lengths on paired end reads. In the interest of accessing new applications through long read technology, nanopore has been investigated for DNA sequencing.

Nanopore sequencing relies on the stable assembly of an insulating membrane layer in which an ion channel is created by protein pore insertion. A single stranded DNA is then forced to translocate through the pore under an applied voltage resulting in different current fluctuations. The fluctuations recorded reach different current levels characteristic of the different nucleotide or series of nucleotides translocating through the pore.

Two promising designs can be considered to create nanopore constructs suitable for DNA nanopore sequencing: 1) biological pores inserted in synthetic (lipidic or polymeric) membranes, and 2) solid-state nanopores (SSNs). Each of these technologies has its own challenges. For example, biological pores inserted into synthetic membranes often have limited stability. Various strategies have been considered to enhance membrane resistance but this remains a major challenge for commercial product with long shelf-life. Polymeric membranes are more stable than lipid-based membranes. Their stability increases with the length of the polymeric chains. However, a protein pore such as MspA can't be inserted into a thicker membrane due to the hydrophobic mismatch between the thick membrane layer and protein pore. Although SSNs could lead to a very robust product, controlled, consistent and affordable fabrication of ˜1 nm solid-state pores remains a challenge.

### SUMMARY OF THE INVENTION

In some aspects, the disclosure relates to a DNA origami structure for insertion through a membrane, wherein the DNA origami structure includes: a first hydrophilic section at a first end of the DNA origami structure; a stopper section adjacent the first hydrophilic section, wherein the stopper section is configured to lay against the membrane when the DNA origami structure is inserted through the membrane; a second hydrophilic section at a second end of the DNA origami structure; a hydrophobic section between the stopper section and the second hydrophilic section; and an open cavity running through the DNA origami structure from the first end to the second end.

In some embodiments, the DNA origami structure further includes one or more hydrophobic moiety attached to a bottom portion of the stopper section facing the second end. In some embodiments, the one or more hydrophobic moiety is a lipid. In some embodiments, each hydrophobic moiety is covalently attached to a first single stranded DNA that is hybridized with a first single stranded DNA overhang on the DNA origami structure. In some embodiments, the first single stranded DNA overhang includes about 15 to about 30 nucleotides.

In some embodiments, the DNA origami structure further includes one or more hydrophilic moiety attached to a top portion of the stopper section facing the first end. In some embodiments, each hydrophilic moiety is covalently attached to a second single stranded DNA that is hybridized with a second single stranded DNA overhang on the DNA origami structure. In some embodiments, the second single stranded DNA overhang includes about 15 to about 30 nucleotides.

In some embodiments, the DNA origami structure further includes one or more hydrophobic moiety attached to a channel wall inside of the open cavity. In some embodiments, the one or more hydrophobic moiety is covalently attached to a first single-stranded DNA that is hybridized with a first single-stranded DNA overhang inside of the open cavity. In some embodiments, the first single stranded DNA overhang includes about 15 to about 30 nucleotides.

In some aspects, the disclosure relates to a DNA origami structure, wherein the hydrophobic section is about 5 nm to about 20 nm in length. In some embodiments, the stopper section is about 20 nm to about 150 nm in width. In some embodiments, the DNA origami structure is about 10 nm to about 150 nm in length. In some aspects, the open cavity is configured to retain a protein pore structure. In some embodiments, the open cavity has a width of about 5 nm to about 25 nm.

In some aspects, the disclosure relates to a stable nanopore construct includes a DNA origami structure and a protein pore immobilized in the open cavity of the DNA origami structure. In some embodiments, the protein pore is covalently linked to the DNA origami structure through a plurality of chemistries including but not limited to thiol modifications, photocrosslinkers, amide bond formation, azide/DBCO (Dibenzocyclooctyne), tetrazines, norbornenes. In some embodiments, through thiol modifications, 3′ thiol Modifier C3 S-S, thiol Modifier C6 S-S, 5′ Amino Modifier C6, 5′ Amino Modifier C12, 5′ Dithiol, aziridine modification, Ni-NTA, DBCO/azide. In some embodiments, the protein pore is a MspA pore.

In some aspects, the disclosure relates to a stable nanopore construct, further including a membrane through which the DNA origami structure is inserted. In some embodiments, the membrane is a polymer membrane, a lipid membrane, a solid-state membrane, or a solid-state nanopore membrane. In some embodiments, the solid-state membrane includes an aperture where the DNA origami structure is inserted through, the aperture has a diameter of about 20 nm to about 50 nm. In some embodiments, the disclosure relates to a stable nanopore construct, wherein the solid-state membrane further includes at least one molecular catch capable of binding to the DNA origami structure.

In some aspects, the disclosure relates to a method for determining a sequence of a polynucleotide using the stable nanopore constructed disclosed herein under an applied voltage; measuring current fluctuations as the polynucleotide passes through the stable nanopore construct; and identify bases of the polynucleotide based on the current fluctuations.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A illustrates a non-limiting example of one possible DNA origami structure. FIG. 1B schematically illustrate a top-down view of a non-limiting example of a DNA origami structure, showing a cavity opening and a stopper section of the DNA origami structure.

FIG. 2A depicts a top-down view of a non-limiting example of a DNA origami structure having the inside of the open cavity-functionalized with hydrophobic moieties.

FIG. 2B illustrates a side view of a non-limiting example of a DNA origami structure having the inside of the open cavity-functionalized with hydrophobic moieties.

FIG. 2C illustrates a non-limiting example of a DNA origami structure having ssDNA overhangs on the top and bottom portions of the stopper section.

FIG. 3A illustrates a non-limiting example of a DNA origami structure inserted in a membrane.

FIG. 3B illustrates the length and the width measurements for each section of a non-limiting example of a DNA origami structure.

FIG. 4 illustrate the dimensions of a MspA protein pore.

FIGS. SA and 5B illustrate two embodiments of the DNA origami structures wherein the width of the open cavity can vary to fit a protein pore.

FIG. 5C illustrates another non-limiting example of a DNA origami structure inserted in a membrane. White circles are a generic representation of a hydrophobic modification for example but not limited to cholesterol modified oligonucleotides. Black circles represent fluorescent labelled molecules or any other tag molecules for example biotin functionalized oligonucleotides. Crossed circles represent oligonucleotides functionalized with a plurality of reactive groups that can be covalently attached to a target protein that is in its proximity. Reactive functional groups include but not limited to photo crosslinkers, N-hydroxysuccinimide (nhs) carboxylates, thiols, etc.

FIG. 6A schematically illustrates a non-limiting example of a nanopore construct with the DNA origami structure inserted into a lipid membrane. This non-limiting example is not intended to be at scale, nor restricted to any measurement.

FIG. 6B schematically illustrates a non-limiting example of a nanopore construct with the DNA origami structure inserted into a polymer membrane. The interior of the upper part of the cavity of the DNA origami is modified with reactive groups that serve to attach the protein nanopore to its cavity. The reactive groups have a plurality of functional groups, thiols, aldehydes, aziridines, azides, etc.

FIG. 6C schematically illustrates a second design of a non-limiting example of a nanopore construct with the DNA origami structure inserted into a polymer membrane. The interior of the upper aperture in the DNA origami is decorated with a variety of hydrophobic terminal groups that enable the insertion of the protein nanopore. This groups include but are not limited to lipid functionalized oligonucleotides, block co-polymer functionalized oligonucleotides, hydrophobic polymer functionalized oligonucleotides, etc. The hydrophobic layer also serves as an insulating layer blocking the free passage of ions and related hydrophilic molecules.

FIG. 7 schematically illustrates a picture of a solid-state nanopore.

FIG. 8 schematically illustrates a non-limiting example of a nanopore construct with the DNA origami structure inserted into a solid-state nanopore.

### DETAILED DESCRIPTION

To provide stable nanopore construct that can withstand higher currents and higher mechanical stress, a DNA origami structure may be utilized with a biological nanopore to form an ion channel through the membrane. DNA origami are 2D and 3D nanoscale structures formed by controlled folding of multiple DNA strands (strapples) and long viral template ssDNA (or circular). In some embodiments, DNA origami structure can be designed to have a hydrophobic section that match the synthetic membrane hydrophobic domain thickness, thus can be inserted through the membrane. Since the length of the hydrophobic section in the DNA origami structure is tunable, it can be used with various synthetic membrane thicknesses without encountering any issue with hydrophobicity mismatch associated with thicker membranes. The biological nanopore is then embedded inside of the larger DNA origami structure to form a nanopore construct for insertion into a synthetic membrane, eliminating membrane thickness restriction due to the size mismatch.

Solid-state nanopores with openings around a 1 nm range can also be used for sequencing. However, it can be difficult to reproducibly fabricate and mass-produce solid-state nanopores (SSNs) with such small cavities. Since a solid-state membrane with a larger opening is easier to make, in some embodiments, the DNA origami structure may be used as a “size reducer” for 20-50 nm SSNs. In some embodiments, once the DNA origami structure is inserted into the SSNs, a protein pore can then be added to the DNA origami structure. In other embodiments, the biological nanopore may be inserted into the DNA origami structure before inserting the DNA origami structures into the membrane.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “membrane” refers to a non-permeable or semi-permeable barrier or other sheet that separates two liquid/gel chambers (e.g., a cis well and a fluidic cavity or reservoir) which can contain the same compositions or different compositions therein. The permeability of the membrane to any given species depends upon the nature of the membrane. In some examples, the membrane may be non-permeable to ions, to electric current, and/or to fluids. For example, a lipid membrane may be impermeable to ions (i.e., does not allow any ion transport therethrough), but may be at least partially permeable to water (e.g., water diffusivity ranges from about 40 μm/s to about 100 μm/s). For another example, a synthetic/solid-state membrane, one example of which is silicon nitride, may be impermeable to ions, electric charge, and fluids (i.e., the diffusion of all of these species is zero). Any membrane may be used in accordance with the present disclosure, as long as the membrane can include a transmembrane nanoscale opening and can maintain a potential difference across the membrane. The membrane may be a monolayer or a multilayer membrane. A multilayer membrane includes two or more layers, each of which is a non-permeable or semi-permeable material.

The membrane may be formed of materials of biological or non-biological origin. A material that is of biological origin refers to material derived from or isolated from a biological environment such as an organism or cell, or a synthetically manufactured version of a biologically available structure (e.g., a biomimetic material)

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture that is substantially perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

A material that is not of biological origin may also be used as the membrane. Some of these materials are solid-state materials and can form a solid-state membrane, and others of these materials can form a thin liquid film or membrane, such as but not limited to a polymeric material. The solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In addition, thesolid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite. Examples of suitable thin liquid film materials that may be used include diblock copolymers or triblock copolymers, such as amphiphilic PMOXA-PDMS-PMOXA ABA triblock copolymers.

The application of an electric potential difference across a nanopore may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the constriction, for example. The signal from that change in current can be measured using any of a variety of methods. Each signal is unique to the species of nucleotide(s) (or probe or linker constructs with a reporter barcode region) in the nanopore, such that the resultant signal can be used to determine a characteristic of the polynucleotide. For example, the identity of one or more species of nucleotide(s) (or probe) that produces a characteristic signal can be determined.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 0.5 nm to about 100 nm, or to less than 1000 nm). Examples of the nanopore include, for example, biological nanopores, solid-state nanopores, and biological and solid-state hybrid nanopores.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, biopolymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, α-helix bundle. nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, aerolysin, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore. MspA is also an example of a protein pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated. In some embodiments, the protein nanopore is modified. This modification can include the attachment of a function group to the inside or the outside of the protein nanopore, such as but not limited to thiol modifications. In some examples, the modification confers an addition function to the nanopore, such as the ability to interact with another protein or membrane.

Also as used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride nanopores, silicon dioxide nanopores, and graphene nanopores.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, the term “single-stranded,” when used in reference to a polynucleotide, means that essentially none of the nucleotides in the polynucleotide are hydrogen bonded to a respective nucleotide in a complementary polynucleotide.

The terms top, bottom, lower, upper, on, etc. are used herein to describe the nanopore construct and/or the DNA origami structure. It is to be understood that these directional terms are not meant to imply a specific orientation, but are used to designate relative orientation between components. The use of directional terms should not be interpreted to limit the examples disclosed herein to any specific orientation(s). As used herein, the terms “upper”, “lower”, “vertical”, “horizontal” and the like are meant to indicate relative orientation.

As used herein, by “translocation,” it is meant that an analyte (e.g., DNA) enters one side of an opening of a nanopore and move to and out of the other side of the opening. It is contemplated that any embodiment herein comprising translocation may refer to electrophoretic translocation or non-electrophoretic translocation, unless specifically noted. An electric field may move an analyte (e.g., a polynucleotide) or modified analyte. By “interacts,” it is meant that the analyte (e.g., DNA) or modified analyte moves into and, optionally, through the opening, where “through the opening” (or “translocates”) means to enter one side of the opening and move to and out of the other side of the opening. Optionally, methods that do not employ electrophoretic translocation are contemplated. In some embodiments, physical pressure causes a modified analyte to interact with, enter, or translocate (after alteration) through the opening. In some embodiments, a magnetic bead is attached to an analyte or modified analyte on the trans side, and magnetic force causes the modified analyte to interact with, enter, or translocate (after alteration) through the opening. Other methods for translocation include but not limited to gravity, osmotic forces, temperature, and other physical forces such as centripetal force.

The aspects and examples set forth herein and recited in the claims can be understood in view of the above definitions.

### DNA Origami Structure

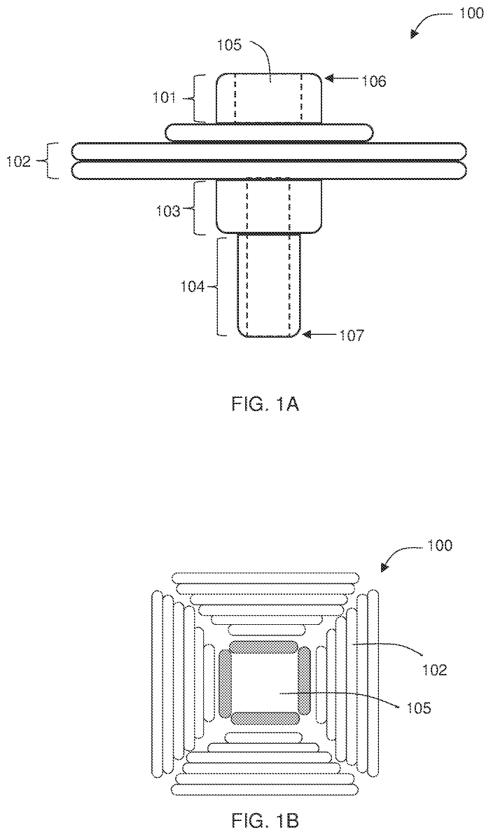

Disclosed herein is a DNA origami structure configured for insertion through a membrane. FIG. 1A depicts a side view of an example of the DNA origami structure 100, and FIG. 1B is a top view of the DNA origami structure 100. The DNA origami structure 100 comprises a first hydrophilic section 101 at a first end 106 of the DNA origami structure 100, a stopper section 102 adjacent the first hydrophilic section 101, wherein the stopper section 102 is configured to lay against the membrane 110 when the DNA origami structure 100 is inserted through the membrane 110, a second hydrophilic section 104 at a second end 107 of the DNA origami structure 100, a hydrophobic section 103 between the stopper section 102 and the second hydrophilic section 104, and an open cavity 105 running through the DNA origami structure 100 from the first end 106 to the second end 107.

The membrane 110 may be a synthetic membrane, such as a lipidic or a polymeric membrane, or a solid-state membrane. In some embodiments, the membrane may be hydrophilic. In some embodiments, the membrane is amphipathic. In some embodiments, the membrane is hydrophobic. In some embodiments, the membrane comprises lipid. In some embodiments, the membrane comprises phospholipid. In some embodiments, the membrane is a lipid bilayer. In some embodiments, the membrane is a phospholipid bilayer. In some embodiments, the membrane is a polymer membrane. For example, the membrane may be a block-copolymer bilayer. In some embodiments, the membrane is a solid-state membrane. Non-limiting examples of solid-state membrane may comprise silicon nitride, silicon oxide, aluminum oxide, hafnium oxide, metal oxide (e.g., tin oxide, gallium oxide, indium oxide, etc.), or 2D materials such as graphene, transition metal dichalcogenide (e.g., MoS₂, WS₂, etc.), or borophene. The solid-state membrane may comprise an aperture where the DNA origami structure may be inserted through. In some embodiments, the aperture has a diameter of about 5 nm to about 100 nm, about 10 to about 90 nm, or about 20 nm to about 70 nm. The thickness of the solid-state membrane may range from about 0.3 nm to about 30 nm. In some embodiments, silicon nitride, silicon oxide, aluminum oxide, and hafnium oxide membranes may have a thickness between about 10 nm and about 30 nm. In some embodiments, metal oxide membranes may have a thickness of between about 1 nm and about 3 nm. In some embodiments, 2D material membranes may have a thickness between about 0.3 nm and about 1 nm.

In some embodiments, the DNA origami structure can be functionalized to further comprises one or more hydrophobic moiety attached to a bottom portion of the stopper section facing the second end of the DNA origami structure. The hydrophobic moieties are attached to the side/portion of the stopper section that should come into contact with the membrane and can facilitate DNA origami structure insertion through the membrane. In some embodiments, the hydrophobic moiety may be a lipid. In some embodiments, the hydrophobic moiety may be cholesterol, tocopherol, solanesol, porphyrin, diglycerol ether, and alkylated phosphate backbone.

In some embodiments, the DNA origami structure can also be functionalized to further comprises one or more hydrophilic moiety attached to a top portion of the stopper section facing the first end of the DNA origami structure. The hydrophilic moieties are attached to the side/portion of the stopper section not laying against the membrane. In some embodiments, the hydrophilic moieties may be PEG azide, PEG aziridine, PEG thiol or any other reactive group.

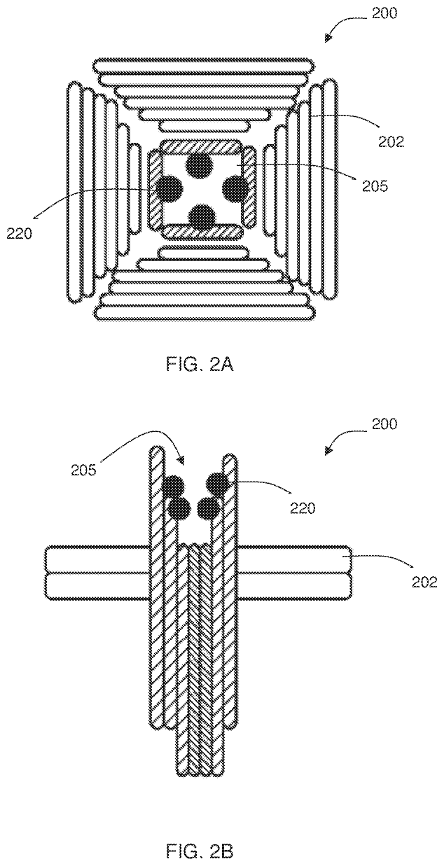

Furthermore, in some embodiments, the channel wall inside of the open cavity may comprised one or more hydrophobic moieties. In some embodiments, the channel wall is functionalized with one or more hydrophobic moieties. FIGS. 2A and 2B depict the hydrophobic moieties 220 inside the open cavity 205. These hydrophobic moieties 220 can facilitate or promote protein pore insertion and/or capture into the open cavity of the DNA. In some embodiments, the hydrophobic moieties 220 inside of the open cavity 205 may promote protein pore insertion in a certain orientation. In some embodiments, the protein pore may further be modified to be covalently attached to the channel wall inside the DNA origami open cavity. In some embodiments, the protein pore is functionalized of have a thiol group that can covalently attach to the channel wall inside of the open cavity, thereby immobilizing the protein pore inside the DNA origami structure.

In some embodiments, the DNA origami structure may further comprise one to fifty single-stranded DNA (ssDNA) overhangs that can support functionalization of the DNA origami structure surface. In some embodiments, the ssDNA overhang may comprise about 15 to about 30 nucleotides. Either hydrophobic or hydrophilic moieties may be conjugated to a ssDNA handle that is complementary to the ssDNA overhang at a certain location on the DNA origami structure. The ssDNA handle can then be hybridized with the ssDNA overhangs to effectively attach the functional moiety to the portion of the DNA origami structure having the corresponding overhangs.

In some embodiments, the portions of DNA origami structure where hydrophobic moieties should be attached would have one or more first ssDNA overhangs. For example, as shown in FIG. 2C, the first ssDNA overhangs 211 may be present at the bottom side/portion of the stopper section 202. In some embodiments, the first ssDNA overhangs may also be present in the open cavity. To functionalize the DNA origami structure with hydrophobic moieties, one or more hydrophobic moieties are covalently attached to a ssDNA that is complementary to the first ssDNA overhangs 211, forming a hydrophobic-ssDNA moiety. Then the DNA origami structure is exposed to the hydrophobic-ssDNA moiety. The hydrophobic-ssDNA moiety would hybridize with the complementary first ssDNA overhangs and result in the attachment of the hydrophobic moieties to the desired portion of the DNA origami structure. In some embodiments, detergent in low concentration can be introduced into the system to avoid any possible aggregation of the hydrophobic-ssDNA moieties.

In some embodiments, the portions of DNA origami structure where hydrophilic moieties should be attached would have one or more second ssDNA overhangs. For example, as seen in FIG. 2C, the second ssDNA overhangs 212 may be present at the first side/portion of the stopper section 202. To functionalize the first portion of the DNA origami structure with hydrophilic moieties, one or more hydrophilic moieties are covalently attached to a first ssDNA that is complementary to the second ssDNA overhangs on the DNA origami structure, forming a hydrophilic-ssDNA moiety. Then the DNA origami structure is exposed to the hydrophilic-ssDNA moiety. The hydrophilic-ssDNA moiety would hybridize with the complementary second ssDNA overhangs and result in the attachment of the hydrophilic moieties to the desired portion of the DNA origami structure.

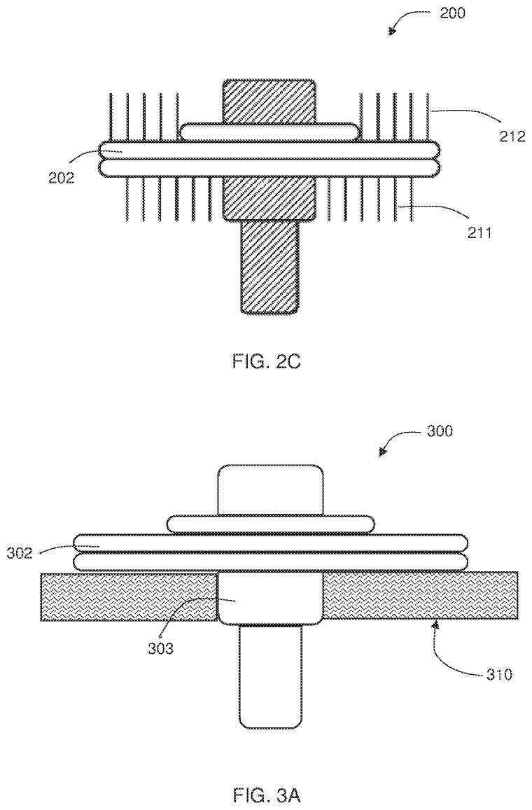

As shown in FIG. 3A, the DNA origami structure 300 is intended to be incorporated into a membrane 310. Thus, the hydrophobic section 303 may vary in length and width such that the hydrophobic section 303 is of the optimal size for interacting with that particular membrane. Similarly, each section of the DNA origami structure may vary in length, as long as that length does not interfere with the overall incorporation of the DNA origami structure into a membrane 310. In some embodiments, with reference to FIG. 3B, the length of the hydrophobic section 303a ranges from about 5 nm to about 100 nm. In some embodiments, the length of the hydrophobic section 303a is about 20 nm, about 30 nm, about 40 nm, about 50 nm, about 60 nm, about 70 nm, or any integer that is between about 20 nm and about 100 nm. In some embodiments, a longer hydrophobic section is more suitable for thicker synthetic membranes. In some embodiments, the length of the hydrophobic section 303a does not need to match the thickness of the solid-state membrane.

In some embodiments, the length of each of the first hydrophilic section 301a and the second hydrophilic section 304a is independently about 1 nm, about 3 nm, about 5 nm, about 10 nm, about 15 nm, about 20 nm, or any integer that is between about 1 nm and about 20 nm. In some embodiments, each of the first hydrophilic section and the second hydrophilic section is independently about 1 nm to about 20 nm, about 3 nm to about 15 nm, or about 5 nm to about 12 nm in length.

In some embodiments, the stopper section 302 may also vary in width 302b to cover the aperture in the membrane (such as an SSN) when the DNA origami structure is inserted in the membrane. In some embodiments, the stopper section 302 also supports the DNA origami structure on the membrane and prevent it from passing through the aperture of the membrane. In some embodiments, the width of the stopper section 302b ranges from about 20 nm to about 150 nm, about 20 nm to about 120 nm, about 30 nm to about 100 nm, or about 40 nm to about 80 nm. In some embodiments, the stopper section width is about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, or about 150 nm, or any integer or range that is between about 20 nm and about 150 nm. In some embodiments, the length of the stopper section 302a is about 2 nm to about 20 nm. In some embodiments, the length of the stopper section 302a is between about 2 nm and about 18 nm, about 3 nm and about 14 nm, about 4 nm and about 10 nm. In some embodiments, the length of the stopper section 302a is about 2 nm, about 5 nm, about 10 nm, about 15 nm, about 20 nm, or any integer or range that is between about 2 nm to about 20 nm.

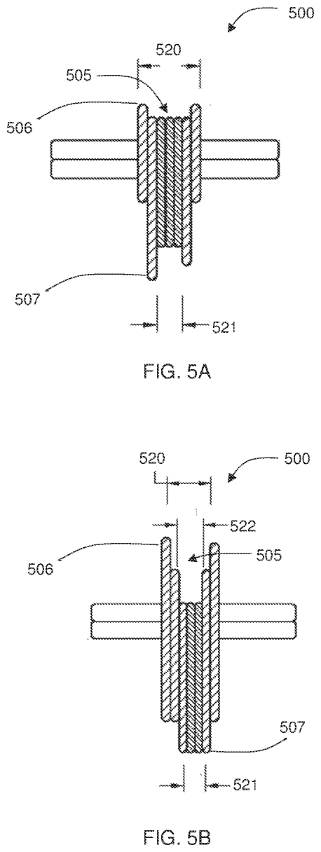

In some embodiments, the open cavity 305 of the DNA origami structure 300 is configured to retain a biological nanopore structure, such as a protein pore. For example, the size of the open cavity 305 is configured to allow the protein pore structure to enter and immobilized within the open cavity 305. In some embodiments, the open cavity has a width 305a of about 5 nm to about 25 nm at the opening. In some embodiments, open cavity 305 has a width 305a of about 5 nm to about 10 nm or about 10 nm to about 25 nm at the opening. In some embodiments, the open cavity has a constant width. In some embodiments, the open cavity has a varying width. In some embodiments, the open cavity 305 may be wider at the opening at the first end of the DNA origami structure, and the open cavity would have a narrower width toward the second end of the DNA origami structure. For example, the open cavity 305 may have a width 305a at the opening at the first end from about 9 nm to about 25 nm, and a width of from about 5 nm to about 8 nm at the second end opening. FIG. 5A depicts one embodiment of such DNA origami structure 500, wherein the open cavity 505 has a first width 520 at the first end opening 506 and a second width 521 extending to the opening at the second end 507. It will be understood that the open cavity can vary in dimensions based upon the size of the stopper section, width of the full DNA origami, protein pore that will be inserted into the open cavity, and size of the nucleotides for sequencing. In some embodiments, the open cavity may have three sections that have different widths. With reference to FIG. 5B, the open cavity 505 has a first section with a width 520 at the first end opening 506, a second section with a width 521 at the second end opening 507, and a third section between the first end 506 and the second end 507 with a width 522. In some embodiments, the second section can have the smallest width, which may help retain the nanopore that is embedded inside the DNA origami structure.

In some embodiments, the DNA origami structure has a length of about 10 nm to about 150 nm. In some embodiments, the DNA origami structure has a length of about 10 nm, about 20 nm, about 30, nm, about 40 nm, about 50nm, about 60 nm, about 70 nm, about 80 nm, about 90 nm, about 100 nm, about 110 nm, about 120 nm, about 130 nm, about 140 nm, about 150 nm or any ranges in between.

### Stable Nanopore Construct

Also disclosed herein is a stable nanopore construct comprising a protein pore inserted into a DNA origami structure. The nanopore construct is stable because the use of DNA origami structure allows the use of thicker membranes due to DNA origami structure's programmable/tunable size. Prior to this, the hydrophobic mismatch between thick membrane layers and protein pores didn't allow the insertion of the protein pores. Inserting DNA origami structure through the membrane enables the utilization of more stable thicker synthetic membranes. The hydrophobic section of the DNA origami can be tuned in order to work with tunable/larger membrane sizes. In some embodiments, once the DNA origami is inserted into the membrane, protein pores can be introduced into the DNA origami structure to allow nanopore sequencing. In some embodiments, the protein pore can be inserted into the DNA origami structure first before the DNA origami structure is inserted through the membrane.

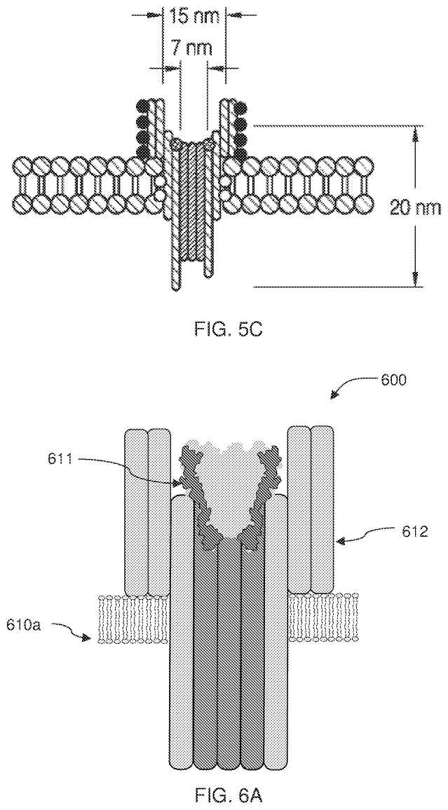

In some embodiments, the stable nanopore construct comprises a DNA origami structure of any one of the embodiments disclosed herein and a pore immobilized in the cavity of the DNA origami structure. As shown in FIG. 6A, in some embodiments, the stable nanopore construct comprises a DNA origami structure of any one of the embodiments disclosed herein and a protein pore immobilized in the cavity of the DNA origami structure. In some embodiments, the protein pore 611 is associated with the open cavity section of the DNA origami structure 612. In some embodiments, the protein pore interacts with the open cavity section of the DNA origami structure through ionic bonds and/or hydrogen bonds.

In some embodiments, the protein pore may comprise at least one modification configured to attach itself to the DNA origami structure inside the open cavity. In some embodiments, as shown in FIG. 6B, the protein pore 611 is covalently linked to the open cavity section of the DNA origami structure 612. In some embodiments, the protein pore is covalently linked to the DNA origami structure through an at least one thiol modification. In some embodiments, some other covalent linkages include photocrosslinkers, amide bond formation, azide/DBCO (Dibenzocyclooctyne), tetrazines, and norbornenes.

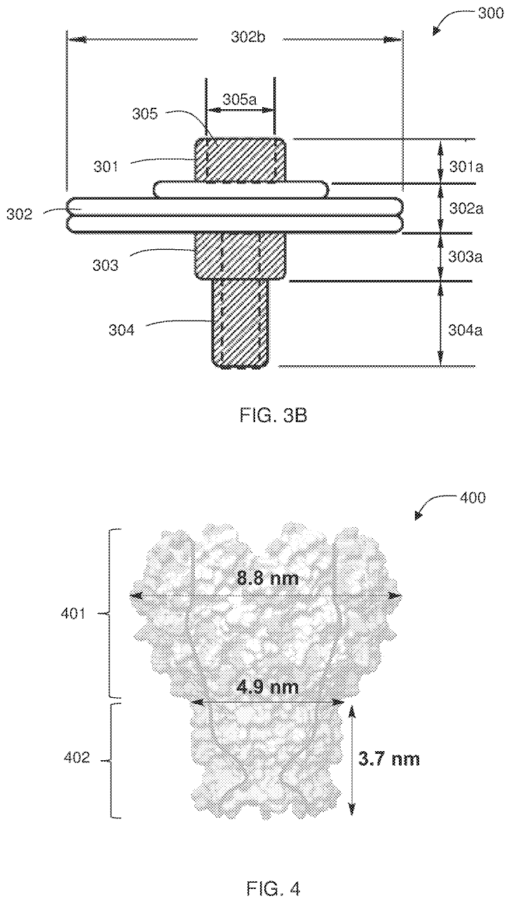

In some embodiments, the DNA origami structure may contain hydrophobic/or amphiphilic support inside of the open cavity where the protein pore may be embedded into (FIG. 6C). In some embodiments, the hydrophobic/or amphiphilic support may be provided by having a hydrophobic moiety conjugated to the first ssDNA that is complementary to and hybridized with the first ssNDA overhang inside of the open cavity. In some embodiments, the protein pore is a MspA pore (FIG. 4).

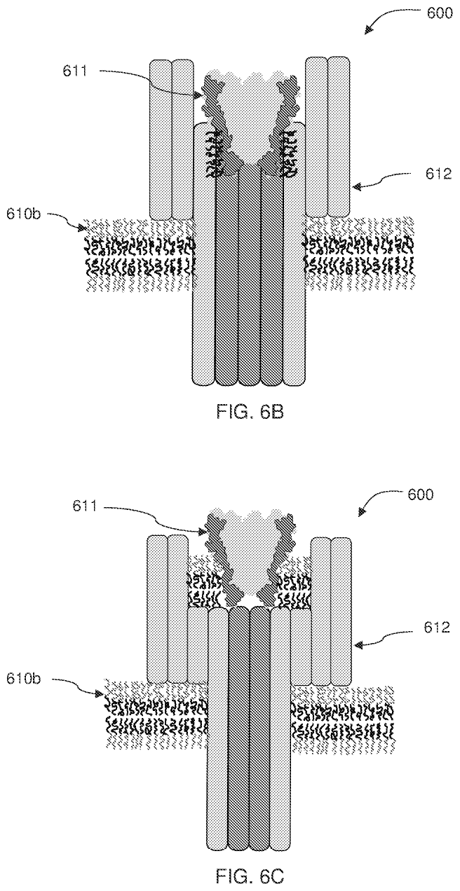

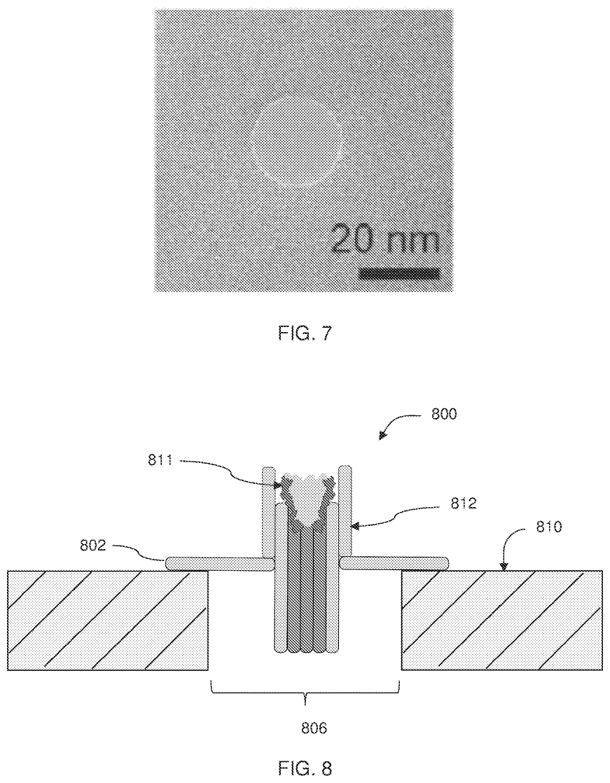

In some embodiments, the stable nanopore construct is directly incorporated into the membrane. In some embodiments, the membrane comprises an aperture through which the stable nanopore construct is inserted through. As shown in FIG. 7, in some embodiments, the solid-state membrane comprises an aperture where the stable nanopore construct is inserted through. In some embodiments, the aperture has a diameter of about 20 nm, about 30 nm, about 40 nm, about 50 nm, about 60 nm, about 70 nm, 80 nm, about 90 nm, about 100 or any integer or any range that is between about 20 nm to about 100 nm, in width. In some embodiments, the aperture has a diameter of about 20 nm to about 50 nm or about 20 nm to about 40 nm. In some embodiments, DNA origami structures are used to fill the size gap between the larger SSN and a smaller MspA protein pore structure that is about 5 to about 8 nm in width. For example, as shown in FIG. 8, the stable nanopore construct 800 is inserted into the aperture 806 on a solid-state membrane 810. The stopper section 802 of the DNA origami 812 is sized larger enough to cover the aperture and provide support for the stable nanopore construct 800. The nanopore 811 is embedded in the open cavity of the DNA origami structure. The shape of the stopper section is not particularly limited. In some embodiments, the stopper section may have a squared shape. In some embodiments, the stopper section may have a circular, pentagonal, or octagonal shape.

In some embodiments, moieties that can interact with the solid-state membrane may be covalently linked to the first ssDNA that is complementary to the first ssDNA overhangs on the DNA origami structure, such as those on the bottom portion of the stopper section. Such moieties can then be attached to the DNA origami structure for securing the DNA origami structure to the solid-state membrane. The moieties include a variety of functional groups including but not limited to thiol functional groups. In some embodiments, the DNA origami structure further comprises a passivation layer to prevent nonspecific binding. Non-limiting examples of a passivation layer include those created through surface modifications, such as silanization, and those created through deposition, such as polymer coating, L-b-L assemblies, polymeric nano-discs, and a polymer bilayer.

In some embodiments, the insertion of the biological nanopore into the DNA origami structure can be done prior to the assembly of the DNA origamis into the membranes. Modifying the inside of the open cavity of the DNA origami structure with hydrophobic moieties may help guide or assist the biological nanopore to go into open cavity.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, embodiment embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

1. A DNA origami structure for insertion through a membrane, wherein the DNA origami structure comprises:
a first hydrophilic section at a first end of the DNA origami structure;
a stopper section adjacent the first hydrophilic section, wherein the stopper section is configured to lay against the membrane when the DNA origami structure is inserted through the membrane;
a second hydrophilic section at a second end of the DNA origami structure;
a hydrophobic section between the stopper section and the second hydrophilic section; and
an open cavity running through the DNA origami structure from the first end to the second end.

2. The DNA origami structure of claim 1, further comprising one or more hydrophobic moieties attached to a bottom portion of the stopper section facing the second end.

3. The DNA origami structure of claim 2, wherein each hydrophobic moiety is covalently attached to a first single-stranded DNA that is hybridized with a first single-stranded DNA overhang on the DNA origami structure.

4. The DNA origami structure of claim 1, further comprising one or more hydrophobic moieties attached to a channel wall inside of the open cavity.

5. The DNA origami structure of claim 4, wherein the one or more hydrophobic moieties is covalently attached to a first single-stranded DNA that is hybridized with a first single-stranded DNA overhang inside of the open cavity.

6. The DNA origami structure of claim 2, wherein the one or more hydrophobic moieties is cholesterol or tocopherol.

7. The DNA origami structure of claim 3, wherein the first single-stranded DNA overhang comprises about 15 to about 30 nucleotides.

8. The DNA origami structure of claim 1, further comprising one or more hydrophilic moieties attached to a top portion of the stopper section facing the first end.

9. The DNA origami structure of claim 8, wherein each hydrophilic moiety is covalently attached to a second single-stranded DNA that is hybridized with a second single-stranded DNA overhang on the DNA origami structure.

10. The DNA origami structure of claim 1, wherein the membrane is a polymer membrane, a lipid membrane, or a solid-state membrane.

11. The DNA origami structure of claim 1, wherein the hydrophobic section is about 5 nm to about 100 nm in length.

12. The DNA origami structure of claim 1, wherein the stopper section is about 20 nm to about 150 nm in width.

13. The DNA origami structure of claim 1, wherein the open cavity is configured to retain a protein nanopore.

14. The DNA origami structure of claim 13, wherein the open cavity has a width of about 5 nm to about 15 nm.

15. The DNA origami structure of claim 1, wherein the stopper section is about 2 nm to about 20 nm in length.

16. The DNA origami structure of claim 1, wherein the DNA origami structure is about 10 nm to about 150 nm in length.

17. A stable nanopore construct comprises a DNA origami structure of claim 1 and a protein pore immobilized in the open cavity of the DNA origami structure.

18. The stable nanopore construct of claim 17, wherein the protein nanopore is covalently linked to the DNA origami structure through thiol modifications, 3′ thiol Modifier C3 S-S, thiol Modifier C6 S-S, 5′ Amino Modifier C6, 5′ Amino Modifier C12, 5′ Dithiol, aziridine modification, Ni-NTA, DBCO/azide.

19. The stable nanopore construct of claim 17, wherein the protein nanopore is a MspA pore.

20. The stable nanopore construct of claim 19, further comprising a membrane through which the DNA origami structure is inserted.

21. The stable nanopore construct of claim 20, wherein the membrane is a polymer membrane, a lipid membrane, or a solid-state membrane.

22. The stable nanopore construct of claim 21, wherein the membrane is a solid-state membrane comprising an aperture where the DNA origami structure is inserted through, the aperture has a diameter of about 5 nm to about 100 nm.

23. A method for determining a sequence of a polynucleotide, the method comprising:
translocating the polynucleotide through a stable nanopore construct of claim 17 under an applied voltage;
measuring current fluctuations as the polynucleotide passes through the stable nanopore construct; and
identify bases of the polynucleotide based on the current fluctuations.
